(19) United States
(12) Patent Application Publication
(10) Pub. No.: US 2025/0295216 A1
(43) Pub. Date: Sep. 25, 2025

(54) Temperature Controlled Applicator For Cosmetic Product

(71) Applicant: ELC MANAGEMENT LLC, Melville, NY (US)

(72) Inventors: Matthew Morris Chateauvert, Port Chester, NY (US); Wilson A. Lee, Hauppauge, NY (US); David Edward Wilson, Plainview, NY (US)

(21) Appl. No.: 18/614,186

(22) Filed: Mar. 22, 2024

(51) Int. Cl. A45D 40/26

(52) U.S. Cl. CPC A45D 40/26; A45D 2200/10; A45D 2200/155; A45D 2200/207

(57) ABSTRACT

A handheld device for applying a cosmetic substance to skin has a housing capable of to be grasped by a user, a head main body portion coupled to the elongated portion, a head main body operably coupled to the housing, at least one cooling element, and an applicator tip positioned at or near the cooling element such that the cooling element removes thermal energy from the applicator tip. A kit may include the handheld tool for applying a cosmetic substance and one or more chips comprising the cosmetic substance to be applied to the skin. The one or more chips may include at least one microneedle composed of the cosmetic substance to be applied to the skin. The handheld device of the system may be operable to insert the at least one microneedle into an epidermal layer of the skin.

### FIELD OF THE DISCLOSURE

The present disclosure generally relates to cosmetic, hair care, body care, and/or skincare products and, more particularly, to systems, devices, and approaches for applying such products.

### BACKGROUND

Cosmetic and/or skincare products may be provided in a number of forms and may be applied using a number of varying approaches. For example, toner or watery lotion may be applied by spraying or dabbing on with fingers or a cloth pad and applying to skin. Creams may be dispensed from a container and applied with fingers or brushes topically to skin. These conventional methods of storing and dispensing cosmetics and skin care products typically apply cosmetic substances to the uppermost layer of the skin. The active ingredients may reach the deeper layers of the skin in doses which are lower than in the topically applied cosmetic substance. In the case of hygroscopic or moisture sensitive products, storage and the form of the cosmetic substance may affect the efficacy of the active ingredient when the cosmetic substance is applied to a user's skin. For example, granules or anhydrous micro-structures stored with desiccants may ensure the stability of a product longer than a cream stored in a tub or a liquid in a bottle that is exposed to ambient air. Further, the conventional methods applying cosmetic substances can lack precise control over the area affected by the active ingredients, potentially also leading to wasted product.

Systems exist that utilize microneedles for applying therapeutic substances intradermally. Such systems utilize a spring-loaded pressure switch to activate an applicator mechanism to ensure that a minimum force threshold is exceeded so that the microneedles penetrated into the skin to at least a desired depth. For these systems, there is no existing mechanism on the device which prevents a user from applying enough force to damage tissues under their skin.

Accordingly, there is a need for systems with improved functionalities for applying cosmetic substances.

### SUMMARY

Examples within the scope of the present disclosure are directed to a handheld device for applying a cosmetic substance to skin. The handheld device includes a housing capable of be grasped by a user, a head main body, at least one cooling element, and an applicator tip. The head main body is operably coupled with a first end of the housing. The head main body includes an inner region defining a cavity. The applicator tip is located within the inner region of the head main body. The applicator tip is positioned at or near the at least one cooling element such that the at least one cooling element removes thermal energy from the applicator.

In an approach, the housing is operable to contain a circuit board at the first end of the housing. The handheld tool includes a rechargeable battery in some approaches. The at least one cooling element is a thermoelectric cooling element in some such approaches.

The at least one cooling element is a removeable component operable to be inserted into the handheld tool to remove thermal energy from the applicator tip, and the removeable component has a temperature that is different from the ambient temperature experienced by the handheld tool prior to the insertion of the removable component in some approaches.

In these and other approaches the head main body at least partially surrounds a pressure switch, the pressure switch is at least partially disposed within the inner region of the head main body and including a switch inner region defining a switch cavity, the applicator tip is located at least partially within the switch cavity, and the head main body includes a clearance reducing feature to prevent misalignment of the pressure switch and the applicator tip.

In some such approaches, the clearance reducing feature includes an inner diameter of the head main body and an outer diameter of the pressure switch are sized to be within approximately 120 μm (0.002″ or 0.12 mm). Additionally, or alternatively, in some such approaches, the clearance reducing feature includes multiple ribs on a surface of the inner region of the head main body operable to contact an outer diameter of the pressure switch or to be within approximately 120 μm (0.002″ of 0.12 mm) of contacting the outer diameter of the pressure switch.

In these and other approaches, the applicator tip includes a cosmetic substance receiving member that is operable to accept a chip carrying the cosmetic substance. In such approaches, the cosmetic substance includes at least one of a microcrystalline structure and at least one microneedle disposed on the chip. In some such approaches, the at least one microneedle has a length of between approximately 90 μm and 110 μm. The handheld tool is operable to apply the cosmetic substance such that the microneedle penetrates the skin not beyond a threshold depth, the threshold depth being within an epidermal layer of the skin in some approaches. In such approaches, the threshold depth is approximately 40 μm.

The handheld tool is operable to apply the cosmetic substance by oscillating the applicator tip toward and away from the skin without applying pressure above a threshold level to the skin in some approaches. In these and other approaches, a pressure switch is operable to actuate the applicator tip when the handheld tool is pressed against the skin with a force not greater than the threshold pressure level, in which the pressure switch includes a biasing mechanism and an electronic contact, and in which the biasing mechanism urges the pressure switch in a direct away from the first end of the housing and toward the skin, and additionally in which the electronic contact is operable to interface with a circuit board to cause oscillation of the applicator tip, the circuit board being located at the first end of the housing. Alternatively, or additionally, in some such approaches, the electronic contact includes a sliding contact on an outer diameter of the pressure switch, and the sliding contact interacts with a fixed contact on an inner diameter of the head main body. The fixed contact has two non-conductive zones surrounding a conductive zone, such that the applicator tip will oscillate only when the sliding contact is aligned with the conductive zone of the fixed contact in some such approaches.

Some examples described herein are directed toward a kit for applying a cosmetic substance to skin. The kit includes a handheld tool for applying the cosmetic substance to skin and an array of single-use chips, each chip including a microcrystalline structure of the cosmetic substance. The handheld tool includes a housing capable of be grasped by a user, a head main body operably coupled with a first end of the housing, at least one cooling element, and an applicator tip. The head main body includes an inner region that defines a cavity. The applicator tip is located within the inner region of the head main body and is positioned at or near the at least one cooling element such that the at least one cooling element removes thermal energy form the applicator tip.

In this and other approaches the handheld tool is operable to prevent degradation of the cosmetic substance by cooling the chip. In such approaches, the applicator tip is adapted to carry a chip from the array of single-use chips. The microcrystalline structure is operable to break following insertion of the microcrystalline structure into an epidermal layer of a user's skin in some approaches. In some such approaches, the microcrystalline structure is a microneedle. The cosmetic substance is a polysaccharide in some approaches to the kit.

Examples are described herein that are directed toward a method for applying a cosmetic substance to skin. The method includes attaching a single-use chip comprising the cosmetic substance to an applicator tip of a handheld device for applying a cosmetic substance to skin and pressing a pressure switch of the device against the skin until an applicator tip oscillates in a substantially linear fashion toward and away from the skin, in which the applicator tip of the handheld device is cooled by a cooling element to prevent hydration of the cosmetic substance prior to penetration of the cosmetic substance into an epidermal layer of the skin. The device includes a housing capable of be grasped by a user, a head main body that has an inner region defining a cavity and is operably coupled with a first end of the housing, a pressure switch at least partially disposed within the inner region of the head main body, at least one cooling element, and an applicator tip located within an inner region of the pressure switch. In some such approaches, the applicator tip is positioned at or near the at least one cooling element such that the at least one cooling element removes thermal energy from the applicator tip.

### BRIEF DESCRIPTION OF THE DRAWINGS

The above needs are at least partially met through provision of one, more than one, or any combination of the approaches for systems for containing and dispensing a cosmetic product described in the following detailed description, particularly when studied in conjunction with the drawings, wherein:

FIG. 1 illustrates an exploded view of an example hand-held device for applying a cosmetic substance in accordance with various examples;

FIG. 2 illustrates a cross-sectional view of an applicator tip assembly of the example system of FIG. 1 in accordance with various examples;

FIG. 3 illustrates a cross-sectional view of an applicator tip assembly of an example system in accordance with various examples;

FIG. 4 illustrates a cross-sectional view of an applicator tip assembly of an example system illustrating possible instability in accordance with various examples;

FIG. 5A illustrates a top-down view of an applicator tip assembly of an example system in accordance with various examples;

FIG. 5B a cross-sectional view of an applicator tip assembly of the example system of FIG. 5A taken along the line A-A in accordance with various examples;

FIG. 6A illustrates a top-down view of an applicator tip assembly of an example system in accordance with various examples;

FIG. 6B is a cross-sectional view of an applicator tip assembly of the example system of FIG. 6A taken along the line B-B in accordance with various examples;

FIG. 7 illustrates an array of microstructures, microneedles, comprising a cosmetic substance applied to an epidermal layer of a user's skin using an example system in accordance with various examples;

FIG. 8 illustrates a cross-sectional view of an applicator tip assembly of an example system with a force dependent switch in accordance with various examples;

FIG. 9 illustrates a cross-sectional view of an applicator tip assembly of an example system with a distance sensor in accordance with various examples; and

FIG. 10 illustrates a method for using the systems and apparatus for applying a cosmetic substance in accordance with various examples.

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various examples. Also, common but well-understood elements that are useful or necessary in a commercially feasible examples are often not depicted in order to facilitate a less obstructed view of these various examples. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to these various approaches, apparatuses, kits, and methods for applying a cosmetic substance are provided that allow the cosmetic substance to be administered to an epidermal layer of the user's skin. In some examples, the system may include a handheld tool that includes a portion that is grasped by a user, and a removable or disposable component that allows for utilization of different cosmetic substances. The ability of the system to be used with disposable chips of cosmetic substance can also enable the system to be used with diverse types of cosmetic substances. The systems and devices described herein may be electrically powered, such as battery powered, and the power sources may energize actuators for applicating components, temperature control components, sensors, and instrument status indicators. The systems, handheld tools, kits, and methods of various examples provided herein allow for delivery of cosmetic substances to an epidermal layer of a user's skin with minimal, if any, damage to tissues beneath the epidermis while furnishing the user with the ability to maintain the integrity of the active ingredient of the cosmetic substance until it is applied.

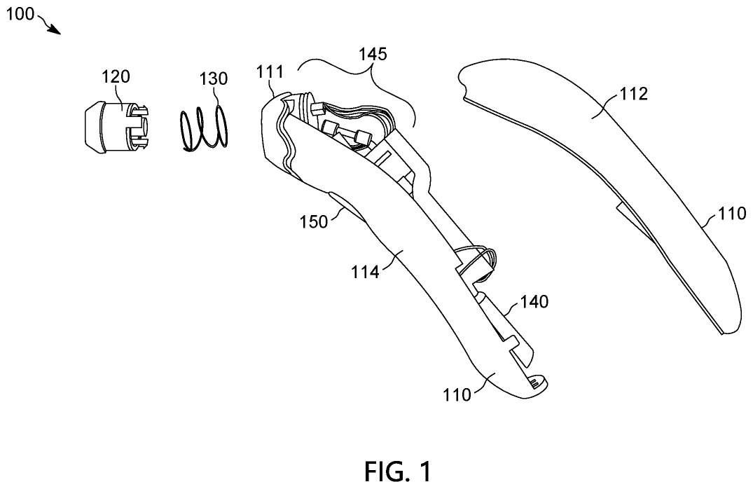

Turning to the Figures, as handheld tool 100 has a housing 110 with a first end 111, a top portion 112, and a bottom portion 114, as well as an applicator tip assembly 120, a spring 130, a power source 140, and electronic components 145, as shown in FIG. 1. The electronic components 145 may include a circuit board (e.g., a mother board, a daughter board, etc.) 180. The housing 110 is capable of being, or operable to be, grasped by a user and has a user interface 150. The user interface 150 may include a power switch, a mode switch, a mode indicator, a status indicator, and the like. The power source 140 may be a battery, including a rechargeable battery.

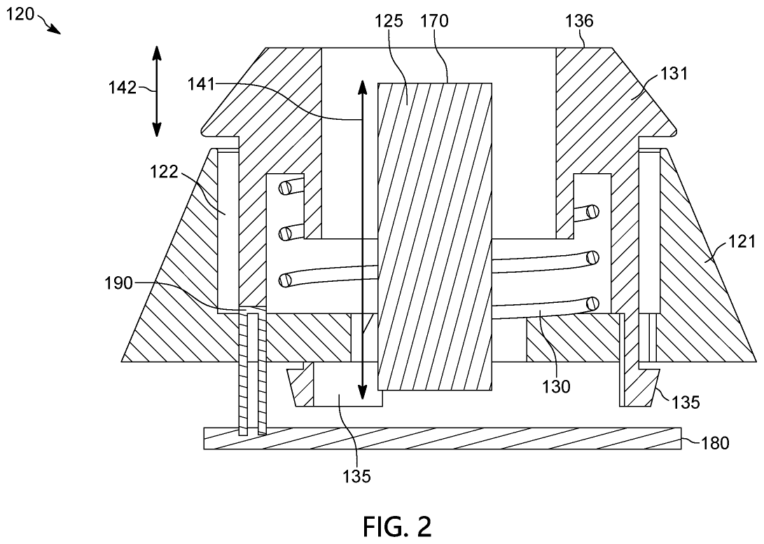

The applicator tip assembly 120, as shown in FIG. 2, includes a head main body 121, an applicator tip 125, a biasing component (e.g., spring) 130, a pressure switch 131, a surface 170 for supporting a chip comprising a cosmetic substance, an interior cavity 122 of the head main body 121, a circuit board 180 located at the first end 111 of the housing 110, and an electrical contact 190 that connects the pressure switch 131 with the circuit board 180.

The head main body 121 includes a an interior volume or interior cavity 122 in which the pressure switch 131 is seated. The pressure switch 131 may include multiple snaps 135 which retain the pressure switch 131 in the interior cavity 122 of the head main body 121. The spring 130 biases, or urges, the pressure switch 131 away from the first end 111 of the housing 110. When the handheld tool is in use, the spring 130 biases the pressure switch 131 toward a user's skin. The pressure switch 131 has a direction of desired motion 142. When the pressure switch 131 moves along the direction of desired motion 142 toward the circuit board 180 such the electrical contact 190 completes a circuit to a certain degree, the applicator tip 125 moves in a direction 141. In the illustrated examples, the applicator tip 125 is located in the innermost portion of the applicator tip assembly 120, in the interior of both the head main body 121 and the pressure switch 131. The applicator tip 125 is shown as having a length extending from the pressure switch 131 toward, but not contacting, the circuit board 180. The motion of the applicator tip 125 along the direction 141 moves the surface 170 that is operable to support a chip with the cosmetic substance to be applied to the skin toward and away from a free end 136 of the pressure switch 131. The free end 136 of the pressure switch 131 is the portion of the pressure switch 131 that is intended to (i.e., operable to) contact the user's skin when the handheld tool is in use. The applicator tip 125 may be driven by an actuator to move in the direction 141. The actuator may include one or more of a linear actuator, a solenoid, a rotary motor, a continuous-drive actuator, a hydraulic actuator, an electric motor, or any other type of actuator suitable for the size and weight constraints of the handheld tool, as well as the power consumption constraints of the handheld tool.

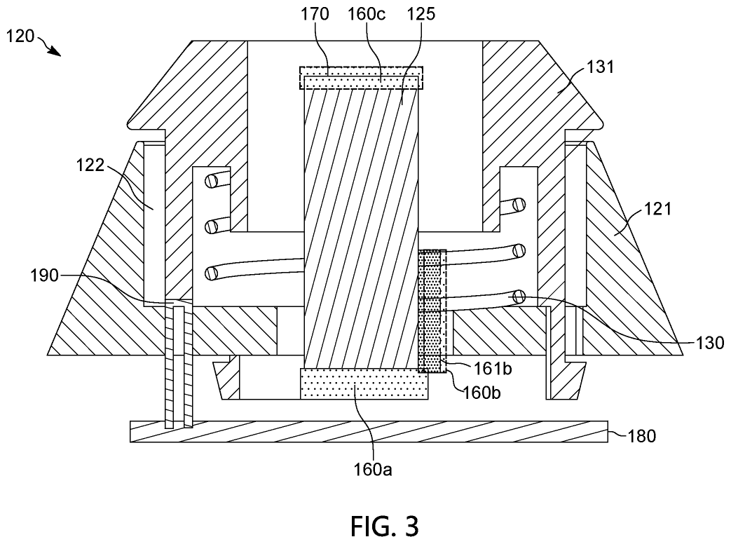

In various examples, the applicator tip assembly 120 includes at least one cooling element 160a, 160b, 160c. FIG. 3 shows various positions for the cooling elements 160a, 160b, 160c within the applicator tip assembly 120. In some configurations, a cooling element 160a is located at the base of the applicator tip 125, either contacting solely the base or surrounding and contacting the base as well as a sidewall portion of the applicator tip 125. An alternate location 161b for a cooling element may be along a bottom portion of the sidewall of the applicator tip. The alternate location 161b is shown as near the base of the applicator tip 125, however the orientation of the cooling element 160b extends toward the middle of the applicator tip 125. A cooling element 160c may also be located at the surface 170 that is operable to support the cosmetic substance when the handheld tool is in use.

The cooling element(s) 160a, 160b, 160c may be of the thermoelectric type. The cooling element(s) 160a, 160b, 160c may be used for heating or cooling. In some approaches to the handheld tool, the user interface 150 may provide a means for setting a temperature or mode for the applicator tip 125. The cooling element(s) 160a, 160b, 160c may cool or heat in response to the temperature or mode selected by a user via the user interface 150. Though not shown in FIG. 3, each cooling element 160a, 160b, 160c has electrical leads leading to the circuit board 180 or to another electrical component in the handheld tool which controls the supply of current to the element 160a, 160b, 160c. The electrical contact 190 which triggers the motion of the applicator tip 125 may be a component in a circuit that includes the cooling element(s) 160a, 160b, 160c.

In various examples, the cooling element 160a, 160b, 160c may be a removeable component that is stored at, or set to, a temperature that may be different from the ambient temperature experienced by the handheld tool prior to the insertion of the cooling element 160a, 160b, 160c into the handheld tool. For example, the cooling element 160a, 160b, 160c may be a hollow cylinder or a hollow tube with a square, rectangular, or other shape in cross-section such that the hollow tube is sized to fit around the applicator tip 125 when inserted into the applicator tip assembly 120. Alternatively, the cooling element 160a, 160b, 160c may be an ingot, partial tube, partial cylinder, sheet of foil, or other shape that is adjacent to, or only partially encircles, the applicator tip 125 when inserted into the applicator tip assembly 120. Transfer of thermal energy between the applicator tip 125, any chip of cosmetic substance at the surface 170 of the applicator tip, and the cooling element 160a, 160b, 160c may occur through contact between elements and components. In some examples, heat transfer may occur convectively, such that the there is no direct contact between the applicator tip 125 and the cooling element 160a, 160b, 160c. In such examples, the ambient air may act as the fluid which facilitates heat transfer between the cosmetic substance, applicator tip 125, and the cooling element 160a, 160b, 160c.

When the cooling element 160a, 160b, 160c is a removable component, the temperature of the cooling element 160a, 160b, 160c prior to insertion into the applicator tip assembly 120 may be set or adjusted by any one or a combination of the following: exposing the cooling element 160a, 160b, 160c to a refrigerated environment, exposing the cooling element 160a, 160b, 160c to a thermoelectric heating or cooling device by direct contact, exposing the cooling element 160a, 160b, 160c to fluid (e.g., air, water, oil) at a desired temperature, and exposing the cooling element 160a, 160b, 160c to electrical current. The cooling element 160a, 160b, 160c may be a metal, a ceramic, a toughened ceramic, a gel in a casing, a solid polymer piece (i.e., a plastic component), a composite material, or any combination thereof. Additionally, the cooling element 160a, 160b, 160c may be a solid with an open structure, may have an exterior structure that is rigid and an interior structure that is liquid or that changes phase, and/or may have a structure which prevents the condensation of moisture directly on the applicator tip. In examples where the cooling element 160a, 160b, 160c has a rigid exterior and a phase changing interior material, the interior material may melt or sublimate when removing thermal energy from the applicator tip 125.

The cooling element 160a, 160b, 160c may serve to remove thermal energy from a chip, with a cosmetic substance, that is affixed to the applicator tip 125 prior to application of the cosmetic substance to skin. The applicator tip 125 accepts a chip or substrate carrying the cosmetic substance, and there may be a cosmetic substance receiving member which accepts or carries the cosmetic substance substrate or chip. The removal of thermal energy from the chip may serve to stabilize the cosmetic substance or an active ingredient of the cosmetic substance. For example, a frozen form of a cosmetic substance may be present on a chip that is affixed to the applicator tip 125 of a handheld tool. In such a case, removing heat from the chip could help to keep the cosmetic substance in the same form, or in approximately the same form, as when it was in storage. Another example is that of a cosmetic substance that is sensitive to ambient relative humidity. Control of the temperature of a chip affixed to the applicator tip 125 may aid in preventing adsorption or absorption of water onto the chip or cosmetic substance, thus preserving the integrity and efficacy of the cosmetic substance. The cooling element(s) 160a, 160b, 160c may affect the temperature of the applicator tip 125 before and during application of the cosmetic substance to an epidermal layer of skin.

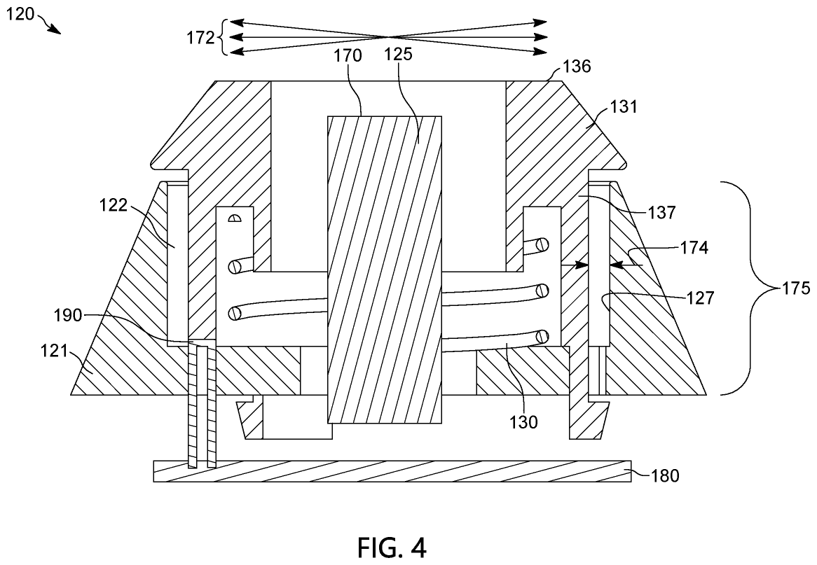

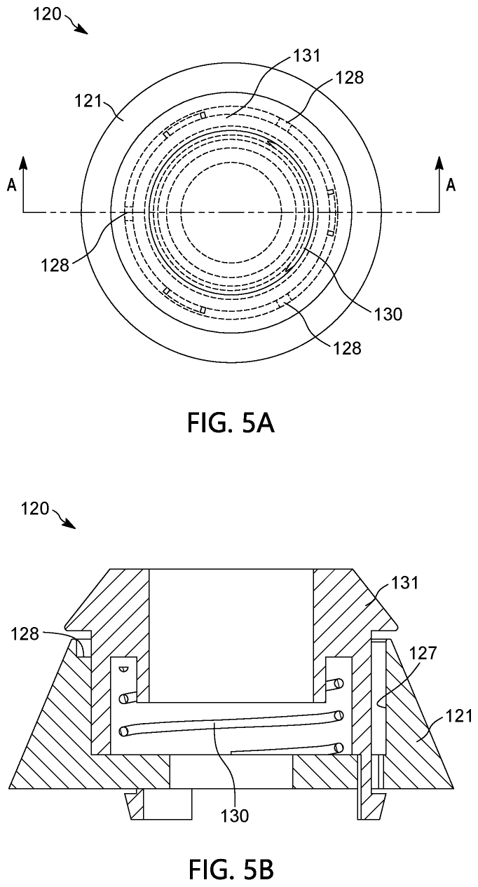

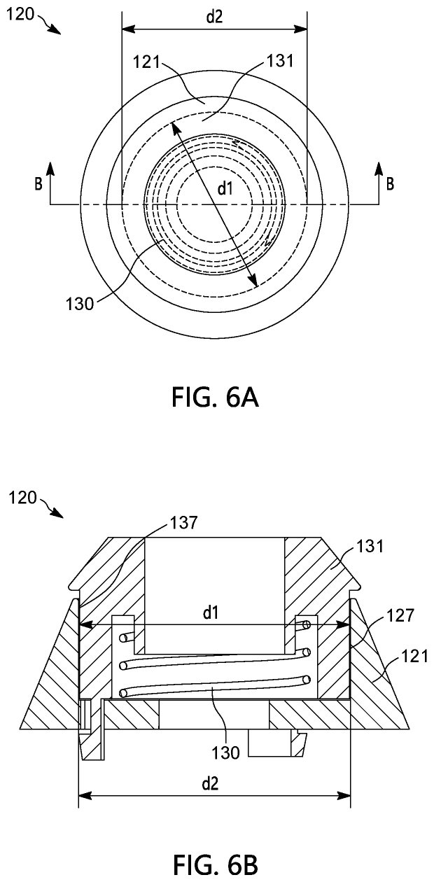

FIG. 4 illustrates directions of unwanted lateral motion 172 in an applicator tip assembly 120, such as that shown in FIGS. 2 and 3. The unwanted lateral motion can cause tilting of the pressure switch 131. This tilting may be visually evident to a user in the free end 136 of the pressure switch 131. Such tilt may cause malfunctioning at the electrical contact 190 or poor delivery of the cosmetic substance mounted at the end surface 170 of the applicator tip 125. The amount of clearance 174 in an area 175 between the pressure switch 131 and the head main body 121 may be a factor in the amount of unwanted lateral motion in the applicator tip assembly 120. The area 175 is shown in FIG. 4 as being the space between an outer wall 137 (e.g., outer sidewall) of the pressure switch 131 and an inner wall 127 of the head main body 121. Various approaches to reducing the clearance 174 is shown in FIGS. 5A-6B and are describe in reference to those figures hereinbelow.

A first approach to reducing the clearance 174 between the pressure switch 131 and the head main body 121 in an applicator tip assembly 120 is shown in FIGS. 5A and 5B. FIG. 5A is a top-down view of the applicator assembly with ribs 128 on the inner wall 127 of the head main body 121. The dotted lines shown in FIG. 5A indicated components which may be obscured from view when the applicator assembly is viewed from the top-down. FIG. 5B is a cross-sectional view of the applicator assembly shown in FIG. 5A taken along the line A-A. In these figures, three ribs 128 are shown, equally spaced along the inner wall 127 of the head main body 121. More than three ribs 128 may be used to reduce the clearance 174 between the pressure switch 131 and head main body 121 with a goal of reducing unwanted lateral motion. Four or more ribs 128 may be used in an applicator tip assembly 120 of a handheld tool.

FIGS. 6A and 6B show another approach to reducing the clearance 174 between the pressure switch 131 and the head main body 121 in an applicator tip assembly 120. FIG. 6A is a top-down view of the applicator assembly with the outer wall 137 of the pressure switch 131 having a first diameter d1 and the inner wall 127 of the head main body 121 having a second diameter d2. FIG. 6B is a cross-sectional view of the applicator assembly shown in FIG. 6A taken along the line B-B. The first diameter d1 is less than or equal to the second diameter d2 to such a degree that the clearance 174 between the outer wall 137 of the pressure switch 131 and the inner wall 127 of the head main body is within a range of between approximately 50 μm and approximately 250 μm (between 0.002″ and 0.010″). The clearance 174 in the applicator tip assembly 120 shown in FIGS. 6A and 6B allows for little tilt in the pressure switch 131 relative to the head main body 121.

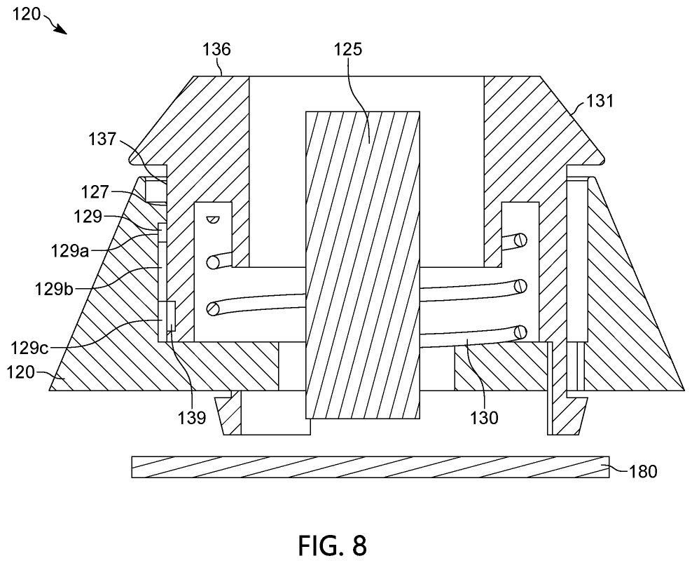

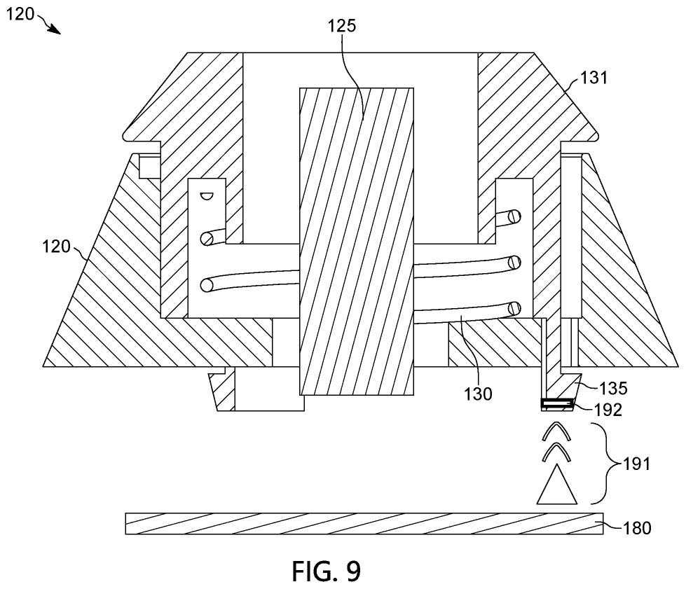

The components and configuration of the applicator tip assemblies 120 shown in FIGS. 5A-6B rely on the structure of the applicator tip assembly 120 itself, particularly the relative sizes of the pressure switch 131 and the head main body 121 which reduce the clearance 174 between these components, to minimize unwanted tilt in the assembly. While excessive tilt may cause the electrical contact 190 to malfunction, in some approaches there may not be any sensors or electrical components which prevent operation of the handheld tool based solely on misalignment of the pressure switch 131 relative to the head main body 121. In other approaches, a sensor which is used to measure distance or relative motion between the pressure switch 131 and the head main body 121, such as shown in FIGS. 8 and 9, may also be used to determine when excessive tilt or unwanted lateral motion occurs in the applicator tip assembly 120 during use. Such a sensor may provide feedback, for example through the user interface 150, or prevent the handheld tool from continuing to function until normal relative motion of the components is restored.

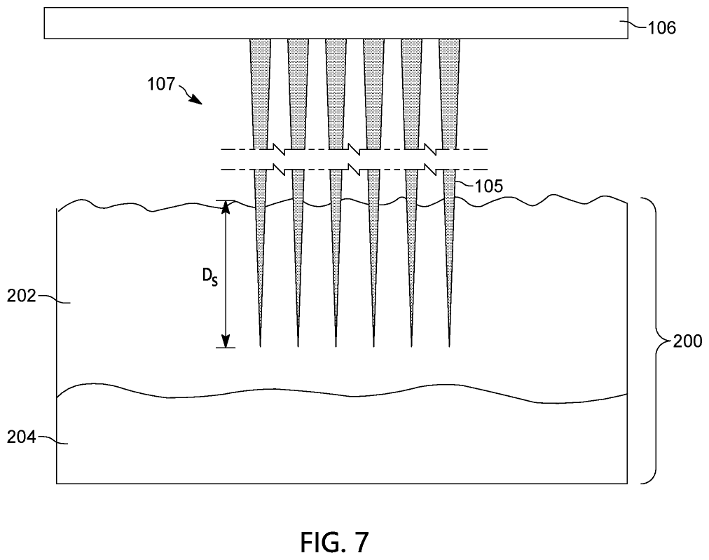

When assembled for use, the surface 170 of the applicator tip will have a cosmetic substance upon it. An applicator tip 125 with cosmetic substance on the surface 170 is shown in FIG. 7. The cosmetic substance is in the form of an array 107 of microstructures supported by a chip or substrate 106. The array of microstructures may be an array of microneedles. The microneedles may be solid or hollow. In some examples, the microstructures are hollow microneedles which allow the cosmetic substance to be delivered to a desired depth into the user's skin by flowing the cosmetic substance through the microneedles. Alternatively, in some examples, the microstructures are solid microneedles that are coated with the cosmetic substance. In other examples, the microstructures are dissolvable microneedles composed of the cosmetic substance. In such examples, where the microneedles are composed of the cosmetic substance, the cosmetic substance may be in a glassy or microcrystalline state, where the cosmetic substance is solid at ambient temperatures (e.g., up to approximately 40 degrees C.) and ambient relative humidity, but once inserted into the epidermis of the skin, the inserted portion of the cosmetic substance microneedle will dissolve, releasing the active ingredient of the microneedle.

FIG. 7 is a schematic showing a chip or substrate 106 supporting an array 107 of microneedles with cosmetic substance 105 which are inserted into skin 200 using a handheld tool 100 in accordance with various examples. The portions of the skin 200 shown are the epidermis 202 and the dermis 204. The microneedles are shown inserted to a depth Dₛ into the epidermis 202. The depth Dₛ may be between approximately 5 μm (micrometers) and approximately 50 μm (micrometers), such as between approximately 10 μm (micrometers) and approximately 45 μm (micrometers), such as between approximately 15 μm (micrometers) and 40 μm (micrometers), or such as between 25 μm (micrometers) and 30 μm (micrometers). The handheld tool 100 may be configured such that the microstructures, or microneedles, penetrate the skin not beyond a threshold depth which is within the epidermal layer of the skin, for example between approximately 5 μm (micrometers) and approximately 50 μm (micrometers), such as between approximately 8 μm (micrometers) and 45 μm (micrometers), including between approximately 10 μm (micrometers) and 40 μm (micrometers). The microstructures (i.e., microneedles) may range in length from about 300 μm (micrometers) to approximately 500 μm (micrometers), such as from about 325 μm (micrometers) to about 475 μm (micrometers), such as from 350 μm (micrometers) to approximately 450 μm (micrometers), and including from about 375 μm (micrometers) to approximately 425 μm (micrometers). The microstructures (i.e., microneedles) may have a base ranging from approximately 50 μm (micrometers) to approximately 100 μm (micrometers), such as from approximately 40 μm (micrometers) to approximately 90 μm (micrometers), such as from approximately 30 μm (micrometers) to approximately 80 μm (micrometers), including from approximately 40 μm (micrometers) to approximately 70 μm (micrometers). In some approaches or implementations, the microneedles may have a length of between approximately 90 μm (micrometers) and 110 μm (micrometers), such as between approximately 95 μm (micrometers) and approximately 105 μm (micrometers), such as between approximately 100 μm (micrometers) and approximately 100 μm (micrometers). The aspect ratio of the microstructures may range from approximately 4:1 to approximately 8:1 (height to base), such as from approximately 9:2 to approximately 15:2 (height to base), including from approximately 5:1 to 7:1 (height to base). For reference, an average human hair has a diameter on the order of approximately 80 μm (micrometers) to approximately 100 μm (micrometers). As mentioned, the microneedles, or microstructures, may be solid or hollow. If hollow, the microstructures may each have a channel of a diameter approximately 0.001 times that of the base of the structure, that is to say on a nanometer scale. Additionally, for hollow microneedles, a reservoir for the cosmetic substance to be applied may be present on the chip or substrate 106. The microstructures, or microneedles, may be conical in shape, or they may have another shape which depends on the materials used in fabricating the microstructure arrays as well as the technique. Other shapes may include columns with sharpened or tapered ends, pyramids, truncated square pyramids, prisms with sharpened or tapered ends, and the like. The sharpened or tapered ends may have a radius of curvature on the order of approximately 1 μm.

It is desirable to deliver the cosmetic substance 105 to the epidermis 202 because there are no nerves in this layer of skin and delivery into the skin in this manner allows for both the preservation of the active ingredients of the cosmetic substance until the moment of use and targeted delivery of the active ingredients. Thus, this method of substance delivery should be more effective than other means of applying the cosmetic substance 105 without being any more painful than conventional application methods. Further, careful delivery of the cosmetic substance 105 to the epidermis 202, prevents injury to deeper layers of the skin and the tissues beneath the skin. Treatment of the skin with the cosmetic substance, or any therapeutic product, without damaging the skin or other tissues is desirable both from an aesthetic perspective, by avoiding discoloration or the creation of wrinkles, and a health perspective. FIGS. 8 and 9 show approaches to an applicator array or applicator tip assembly 120 for a handheld tool for applying a cosmetic substance in which the applicator tip assembly 120 may be prevented from exerting force on a user's skin that exceeds a predetermined threshold value.

FIG. 8 shows a cross-sectional view of an applicator tip assembly 120 with a force dependent switch that includes a first component 129 on the inner wall 127 of the head main body 121 and a second component 139 on the outer wall 137 of the pressure switch 131. The first component 129 has multiple zones. As shown in FIG. 8, the first component 129 has a first zone 129a, as second zone 129b, and a third zone 129c. The first zone 129a is located on a portion of the inner wall 127 of the head main body 121 that is closest to the free end 136 of the pressure switch 131. The second component 139 moves with the pressure switch 131. The location of the second component 139 along the outer wall 137 of the pressure switch is along the base of the wall, on the portion furthest away from the free end 136. When the handheld tool is in use, the pressure switch 131 moves relative to the head main body 121 as a user pushes or moves the tool toward a skin surface. As the second component 139 moves adjacent to the first zone 129a, the applicator tip 125 remains stationary. Once the secondary component 139 moves so that it is adjacent to the second zone 129b, the applicator tip 125 begins moving toward and away from the skin surface in an oscillating motion. The motion of the applicator tip 125 ceases when the second component 139 is adjacent to the third zone 129c of the first component 129. The force dependent switch may include a sliding contact, the second component 139, on an outer diameter of the pressure switch 131, the sliding contact interacting with a fixed contact, the first component 129, on an inner diameter of the head main body, the fixed contact having two non-conductive zones surrounding a conductive zone, such that the applicator tip will oscillate only when the sliding contact is aligned with the conductive zone of the fixed contact.

Motion of the second component 139 in relation to the zones of the first component 129 may cause feedback to the user in the form of status updates to the user interface 150, and additionally, or optionally, feedback to the user may be an audio signal or a physical sensation. The feedback may indicate when the applicator tip 125 will begin and end motion, as well as when the force applied by the user is greater than a lower threshold value (e.g., enough to bring the applicator tip assembly 120 sufficiently in contact with the skin surface) and also when the force applied by the user is greater than an upper threshold value (e.g., a force that may damage the skin or underlying tissue). The handheld tool 100 may be operable to apply the cosmetic substance 105 by oscillating the applicator tip 125 toward and away from the skin at a pressure level at or below a threshold pressure level against the skin, such that the threshold pressure level is approximately just below that which would damage an average user's skin tissue.

FIG. 9 illustrates a cross-sectional view of an applicator tip assembly 120 with a distance sensor 191. The distance sensor 191 may be located on or near the circuit board 180 in the handheld tool. In FIG. 9, the distance sensor 191 is shown as placed beneath one of the snaps 135 on the pressure switch 131. A component 192 on the snap 135 may act as part of the distance sensor 191 in that the component 192 may provide an enhance reflective surface or a more easily detected surface or point. For example, when the distance sensor 191 is based on the strength of a reflected signal, having a reflective surface on the object which is being sensed improves the accuracy of the measurement. As with the applicator tip assembly 120 shown in FIG. 8, the handheld tool may provide feedback based on the relative position of the pressure switch 131 and the head main body 121, or in this case the sensor 191, to indicate to the user when threshold values for minimum and maximum applied force has been reached. Feedback may be provided to the user when the threshold force values have been reached, and the feedback may be in the form of feedback provided via the user interface 150 or haptic (e.g., audio or sensation) feedback.

In use, the handheld tool may be provided along with the cosmetic substance to be applied in a kit. When using the kit, an elongated portion or housing 110 is fitted with a head main body 121. An applicator tip 125 is provided with a cosmetic substance 105 on a first surface 170, with the cosmetic substance 105 being an array 107 of microstructures or microneedles of the cosmetic substance that is supported on a substrate 106. The applicator tip 125 may be preloaded with the substrate 106 and array 107 of cosmetic substance. In such kits, multiple applicator tips may be provided. Alternatively, or additionally, one or two applicator tips 125 may be provided with a kit and an adhesive surface on any of the surface 170 of the applicator tip 125 and the substrate 106 may allow for temporarily fixing the substrate 106 to the applicator tip 125 for use in the system. When ready to use the system, a user places the applicator tip 125 with cosmetic substance 105 into the recess in the head main body 121. Grasping the elongated portion or housing 110, the user presses the applicator tip 125 onto a selected area of skin, driving the each microneedle of the microstructure array 107 to a depth Dₛ into the skin. A feedback mechanism or component may emit a sound, such as a clicking sound, or produce a sensation such as a click or resistive force. The user may cease to drive the elongated portion or housing 110 towards the skin when experiencing the feedback and either hold the system stationary or retract the elongated portion or housing 110. Then, the user may move the system to another area of skin and reapply the applicator tip 125 to the skin using the same array 107 of cosmetic substance containing microstructures. Alternatively, the user may change the applicator tip 125 or substrate (chip) 106 before applying the applicator tip 125 to another area of skin. In some instances, a user may wish to contact the same area of skin with the applicator tip 125 multiple times, either with the same array 107 of cosmetic substance or with a new array 107. In such instances, the user may simply drive the elongated portion or housing 110 toward and away from the skin.

As described herein, the cosmetic substance to be applied to skin may be in the form of an array of microstructures with the cosmetic substance. In some implementations, the microstructures may be microneedles, and the microneedles may be coated with the cosmetic substance, may each have a nanometer scale channel through which the cosmetic substance flows, and/or may be microneedles formed of solidified cosmetic substance. The solidified cosmetic substance may be formed into crystalline microneedles or amorphous, or glassy, microneedles. Alternatively, or additionally, the solidified cosmetic substance may include a frozen suspension of an effective or active ingredient. The array of microstructures may be circular, square, or any other polygon in footprint. There may be one type of microstructure in each array, as in only solid microneedles composed of microcrystalline cosmetic substance, or there may be multiple types of microstructures in each array, such as a combination of solid microneedles coated with cosmetic substance interspersed with solid microneedles composed of microcrystalline cosmetic substance. Other configurations of the arrays and mixture of types of microstructures of cosmetic substance for application to skin are also possible.

Cosmetic substances which can be used with the systems, kits, and methods described herein may include hyaluronic acid, polysaccharides, alpha hydroxy acid, glycolic acid, lactic acid, mandelic acid, citric acid, fatty acids, ceramides, waxy lipid molecules, peptides, and the like. The cosmetic substances may be applied to an epidermis layer of the skin using coated microstructures (microneedles), where the coating includes a gel or dried layer of the cosmetic substance. Alternatively, or additionally, the cosmetic substances may be applied using hollow microstructures or microneedles, laid out in an array on a substrate or chip and where the cosmetic substance flows through the microneedles for delivery to an epidermal layer of the skin. In some implementations, the cosmetic substance may be solidified (e.g., frozen or crystallized) and formed into microstructures supported on a substrate or chip.

The various systems described herein may be provided as a kit including a handheld tool and a chip with a cosmetic substance to be applied to skin. The handheld tool may include a housing 110 and an applicator tip assembly 120, as well as the electronic and power supply components, including the user interface, shown in FIG. 1. The kit may include one or more chips, or substrates, which support cosmetic substance to be applied to skin. The cosmetic substance may be deposited on the substrate as an array of microstructures, such as microneedles, such as described hereinabove.

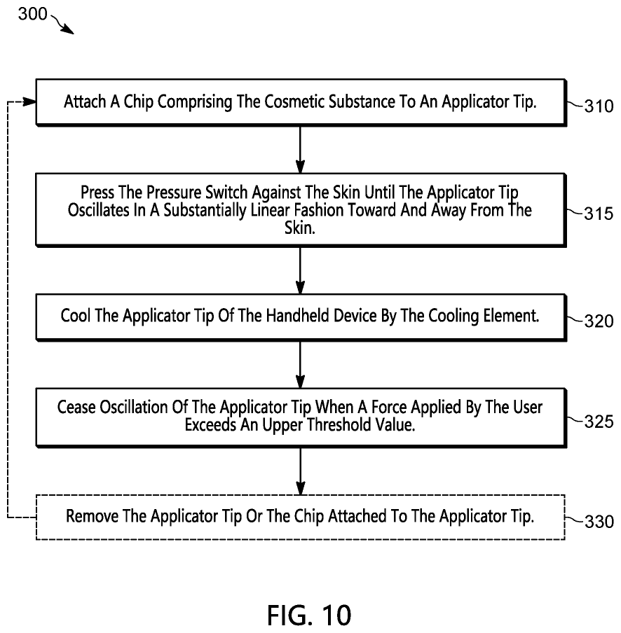

FIG. 10 illustrates a method 300 for using the systems, devices, and kits described herein to deliver a cosmetic substance to skin according to various examples. Systems for use with the method can include a handheld device with a housing operable to be grasped by a user, a head main body operably coupled with a first end of the housing, the head main body including an inner region defining a cavity, a pressure switch at least partially disposed within the inner region of the head main body and which has an inner region defining a cavity, at least one cooling element, and an applicator tip located within the inner region of the pressure switch and which is positioned at or near the cooling element such that the cooling element removes thermal energy from the applicator tip. Such systems can be part of kits which also include one or more chips or substrates with the cosmetic substance to be applied to skin.

In the method, a single-use chip with the cosmetic substance is attached to an applicator tip of a handheld device for applying a cosmetic substance to skin, as in a step 310. The pressure switch is pressed against the skin until the applicator tip oscillates in a substantially linear fashion toward and away from the skin, as in a step 315. This motion of the applicator tip in a linear fashion, substantially perpendicular to the skin may be desirable to optimize delivery of the cosmetic substance to a target portion of the skin. The applicator tip of the handheld device is cooled by the cooling element, as in a step 320. Optionally, the handheld device may cease oscillation of the applicator tip when a force applied by the user exceeds an upper threshold value, as in a step 325. Alternatively, or additionally, the method may include removing the chip attached to the applicator tip, as in a step 330, and further attaching another chip to the applicator tip.

A kit which includes a handheld tool for applying a cosmetic substance and an array of single-use chips made up of the cosmetic substance may be provided. The handheld tool may have features of any of the handheld tools described herein and shown in the figures, including applicator tips and applicator tip assemblies as shown and described. For example, in the kit, the applicator tip of the handheld tool may be adapted to carry a chip from an array of single-use chips in which the handheld tool includes a cooling element to prevent degradation of the cosmetic substance by cooling the chip. The cooling element may cool the applicator tip to prevent hydration of the cosmetic substance prior to penetration of the cosmetic substance into an epidermal layer of the skin. Once in the skin, the cosmetic substance, in the form of microcrystalline structures (e.g., microneedles), may break off in the epidermal layer of the skin.

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings. Additionally, the described embodiments/examples/implementations should not be interpreted as mutually exclusive, and should instead be understood as potentially combinable if such combinations are permissive in any way. In other words, any feature disclosed in any of the aforementioned embodiments/examples/implementations may be included in any of the other aforementioned embodiments/examples/implementations.

The benefits, advantages, solutions to problems, and any element(s) that may cause any benefit, advantage, or solution to occur or become more pronounced are not to be construed as a critical, required, or essential features or elements of any or all the claims. The claimed invention is defined solely by the appended claims including any amendments made during the pendency of this application and all equivalents of those claims as issued.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter may lie in less than all features of a single disclosed embodiment. Thus, the following claims are hereby incorporated into the Detailed Description, with each claim standing on its own as a separately claimed subject matter.

The patent claims at the end of this patent application are not intended to be construed under 35 U.S.C. § 112(f) unless traditional means-plus-function language is expressly recited, such as “means for” or “step for” language being explicitly recited in the claim(s).

What is claimed is:

1. A handheld tool for applying a cosmetic substance to skin, the handheld tool comprising:
a housing capable of being grasped by a user;
a head main body operably coupled with a first end of the housing, the head main body including an inner region defining a cavity;
at least one cooling element; and
an applicator tip located within the inner region of the head main body, the applicator tip positioned at or near the at least one cooling element such that the at least one cooling element removes thermal energy from the applicator tip.

2. The handheld tool of claim 1, wherein the housing is suitable for containing a circuit board at the first end of the housing.

3. The handheld tool of claim 1, wherein the at least one cooling element is a thermoelectric cooling element.

4. The handheld tool of claim 1, wherein the at least one cooling element is a removeable component operable to be inserted into the handheld tool to remove thermal energy from the applicator tip, the removeable component having a temperature that is different from an ambient temperature experienced by the handheld tool prior to insertion of the removable component.

5. The handheld tool of claim 1, wherein the applicator tip comprises a cosmetic substance receiving member that is suitable to accept a chip carrying the cosmetic substance.

6. The handheld tool of claim 5, wherein the cosmetic substance comprises at least one of:
a microcrystalline structure; and
at least one microneedle disposed on the chip.

7. The handheld tool of claim 6, wherein the at least one microneedle has a length of between approximately 90 μm and 110 μm.

8. The handheld tool of claim 7, wherein the handheld tool is suitable to apply the cosmetic substance such that the at least one microneedle penetrates the skin not beyond a threshold depth, the threshold depth being within an epidermal layer of the skin.

9. The handheld tool of claim 8, wherein the threshold depth is approximately 40 μm.

10. The handheld tool of claim 1, wherein the handheld tool is suitable to apply the cosmetic substance by oscillating the applicator tip toward and away from the skin without applying pressure above a threshold pressure level to the skin.

11. The handheld tool of claim 10, wherein a pressure switch is capable of actuating the applicator tip when the handheld tool is pressed against the skin with a force not greater than the threshold pressure level, the pressure switch comprising a biasing mechanism and an electronic contact, wherein the biasing mechanism urges the pressure switch in a direct away from the first end of the housing and toward the skin, and further wherein the electronic contact is capable of interfacing with a circuit board to cause oscillation of the applicator tip, the circuit board being located at the first end of the housing.

12. The handheld tool of claim 11, wherein the electronic contact comprises a sliding contact on an outer diameter of the pressure switch, the sliding contact interacting with a fixed contact on an inner diameter of the head main body, the fixed contact having two non-conductive zones surrounding a conductive zone, such that the applicator tip will oscillate only when the sliding contact is aligned with the conductive zone of the fixed contact.

13. A kit for applying a cosmetic substance to skin, the kit comprising:
a handheld tool for applying the cosmetic substance to skin, the handheld tool comprising:
a housing capable of being grasped by a user;
a head main body operably coupled with a first end of the housing, the head main body including an inner region defining a cavity;
at least one cooling element; and
an applicator tip located within the inner region of the head main body, the applicator tip positioned at or near the at least one cooling element such that the at least on cooling element removes thermal energy from the applicator tip; and
an array of single-use chips, each chip comprising a microcrystalline structure of the cosmetic substance.

14. A method of applying a cosmetic substance onto skin, the method comprising:
attaching a single-use chip comprising the cosmetic substance to an applicator tip of handheld device for applying a cosmetic substance to skin, the handheld device comprising:
a housing capable of being grasped by a user;
a head main body operably coupled with a first end of the housing, the head main body including an inner region defining a cavity;
a pressure switch at least partially disposed within the inner region of the head main body, the pressure switch including an inner region defining a cavity;
at least one cooling element; and
an applicator tip located within the inner region of the pressure switch, the applicator tip positioned at or near the at least one cooling element such that the at least one cooling element removes thermal from the applicator tip; and
pressing the pressure switch against the skin until the applicator tip oscillates in a substantially linear fashion toward and away from the skin,
wherein the applicator tip of the handheld device is cooled by the at least one cooling element to prevent hydration of the cosmetic substance prior to penetration of the cosmetic substance into an epidermal layer of the skin.

15. A handheld tool for applying a cosmetic substance to skin, the handheld tool comprising:
a housing capable of being grasped by a user;
a head main body operably coupled with a first end of the housing, the head main body including an inner region defining a cavity;
a pressure switch, the pressure switch at least partially disposed within the inner region of the head main body and including a switch inner region defining a switch cavity; and
an applicator tip located at least partially within the switch cavity, and further wherein the head main body includes a clearance reducing feature to prevent misalignment of the pressure switch and the applicator tip.

16. The handheld tool of claim 15, wherein the clearance reducing feature comprises an inner diameter of the head main body and an outer diameter of the pressure switch are sized to be within 120 μm.

17. The handheld tool of claim 15, wherein the clearance reducing feature comprises multiple ribs on a surface of the inner region of the head main body suitable to contact an outer diameter of the pressure switch or to be within 120 μm of contacting the outer diameter of the pressure switch.

18. A kit for applying a cosmetic substance to skin, the kit comprising:
a handheld tool for applying the cosmetic substance to skin, the handheld tool comprising:
a housing capable of being grasped by a user;
a head main body operably coupled with a first end of the housing, the head main body including an inner region defining a cavity; and
an applicator tip located within the inner region of the head main body; and
an array of single-use chips, each chip comprising a microcrystalline structure of the cosmetic substance.

19. The kit of claim 18, wherein the applicator tip is adapted to carry a chip from the array of single-use chips, the handheld tool being suitable to prevent degradation of the cosmetic substance by cooling the chip.

20. The kit of claim 18, wherein the microcrystalline structure is suitable to break following insertion of the microcrystalline structure into an epidermal layer.

21. The kit of claim 18, wherein the cosmetic substance is a polysaccharide.

22. A method of applying a cosmetic substance onto skin, the method comprising:
attaching a single-use chip comprising the cosmetic substance to an applicator tip of a handheld device for applying a cosmetic substance to skin, the handheld device comprising:
a housing capable of being grasped by a user;
a head main body operably coupled with a first end of the housing, the head main body including an inner region defining a cavity;
a pressure switch at least partially disposed within the inner region of the head main body, the pressure switch including an inner region defining a cavity; and
an applicator tip located within the inner region of the pressure switch; and
pressing the pressure switch against the skin until the applicator tip oscillates in a substantially linear fashion toward and away from the skin.
